(19) United States
(12) Patent Application Publication
(10) Pub. No.: US 2025/0025403 A1
(43) Pub. Date: Jan. 23, 2025

(54) Film Former

(71) Applicant: Elementis Specialties, Inc., East Windsor, NJ (US)

(72) Inventors: Silke Wiemda Klara Valentin-Burzynksi, Bergheim (DE); Sabine Schäpermeier, Köln (DE); Chun-Hung Yen, Hsinchu City (TW); Ming-Liang Yen, Hsinchu City (TW)

(73) Assignee: Elementis Specialties, Inc., East Windsor, NJ (US)

(21) Appl. No.: 18/222,241

(22) Filed: Jul. 14, 2023

(51) Int. Cl. A61K 8/85; A61K 8/40; A61Q 17/04; C08G 63/40

(52) U.S. Cl. CPC A61K 8/85; A61K 8/40; A61Q 17/04; C08G 63/40

(57) ABSTRACT

The invention relates to a polyester obtained or obtainable by the reaction of one or more linear or branched C₇-C₃₂ mono-alcohols, citric acid, and dipropylene glycol or a polypropylene glycol, and the use of the polyester in a film-forming composition.

### FIELD OF THE INVENTION

The present invention relates to a sunscreen composition. The present invention also relates to a method for the manufacture of a sunscreen composition, and to the use of certain polyesters in sunscreen compositions.

### BACKGROUND

Sunscreen compositions are a well-known type of product in the field of personal care, and are important in protecting human skin from the damaging effects of UV radiation. Sunscreen compositions are available in a number of different formats, such as sprays, lotions, sticks and creams. Sunscreens may provide various levels of sun protection as defined by their SPF (sun protection factor) and may be effective against UV-B and/or UV-A radiation from the sun.

It is well known that the effectiveness of sun-care products such as sunscreens may be reduced as a result of contact with water. For example, sun-care products such as sunscreens may be washed off the skin during swimming, or when the user perspires. This may substantially reduce the effectiveness of the product. For example, during or following contact with water a sunscreen product may be effective for a shorter amount of time, or may be less effective in protecting skin from UV radiation. This may increase the risk of skin damage such as premature aging and carcinogenesis. The presence of sunscreen in bodies of water such as the ocean may also cause environmental damage, for example to coral reefs.

Sun-care products that can provide a level of water resistance are very desirable to consumers. Particularly, it is desirable to maintain reliable sun protection even when in contact with water.

An example of a conventional technology that is included in sunscreens to provide water resistance is microplastic particles. These can increase waterproofing and aid in binding the product ingredients together. However, microplastics are known to be persistent in the environment, and may contribute to water pollution and cause damage to wildlife and human health if ingested.

In addition to the environmental damage that may be caused, many consumers would prefer to use sun-care products that do not include ingredients such as microplastics. Ingredients that are naturally-derived and sustainable may be particularly attractive.

### SUMMARY OF THE INVENTION

The present disclosure provides sunscreen compositions comprising a polyester. It has surprisingly been found that the polyester disclosed herein can address the problems associated with decreasing effectiveness of sunscreen on contact with water. In particular, the polyester disclosed herein can provide increased water resistance to a sunscreen formulation by acting as a film forming composition. The polyester defined herein may be derived from natural ingredients and is biodegradable. Thus, damage to the environment may be minimized.

In a first aspect, the present disclosure provides a polyester obtained or obtainable by the reaction of:
one or more linear or branched C₇-C₃₂ mono-alcohols;
citric acid; and
dipropylene glycol or a polypropylene glycol defined by

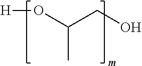

wherein m is from 3 to 35; or mixtures thereof.

A second aspect of the invention provides a polyester having the following formula:

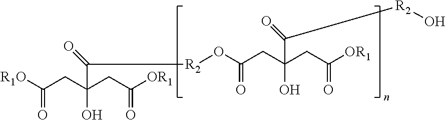

wherein R₁ is a C₇ to C₃₂ linear or branched alkyl group; R₂ is

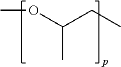

wherein p is from 2 to 35; and where n is from 3 to 105.

In a third aspect, the present disclosure provides a sunscreen composition comprising the polyester as defined herein in the first or second aspect.

It has surprisingly found that the polyester disclosed herein may be useful in enhancing film integrity and water resistance of a sunscreen formulation. This improved film integrity may contribute to enhanced SPF protection. The polyester disclosed herein may provide enhanced film integrity and water resistance in a range of sunscreen formats, including sprays, lotions, sticks and creams. In other words, the film-forming polyester may be capable of forming, either on its own or in the presence of a film-forming aid, a substantially continuous and adherent film on a surface, in particular on the skin.

The polyester disclosed herein can provide film-forming properties to a sunscreen composition. The polyester may act as a film former in various sunscreen systems, and can disperse through the desired system to create a uniform and effective film on the skin. In particular, the polyester may be compatible with multiple chemical classes that may be present in a sunscreen composition. These may include esters, hydrocarbons, natural oils, silicones, ethers, and alcohols.

### DETAILED DESCRIPTION

The following definitions apply to the disclosure herein, including all aspects of the invention and their embodiments.

The compounds and intermediates described herein may be named according to the IUPAC (International Union for Pure and Applied Chemistry) or the CAS (Chemical Abstracts Service) nomenclature systems or by their commercial trade names. It should also be understood that any reference to a compound described herein, such as a polyurea, a primary monoamine, a diamine or a triamine, encompasses all stereoisomers (e.g. cis and trans isomers) and/or optical isomers (e.g. R and S enantiomers) of such compounds, in substantially pure form and/or any mixtures of the foregoing in any ratio.

As used herein by itself or in conjunction with another term or terms, “substituted” indicates that a hydrogen atom on a molecule has been replaced with a different atom or group of atoms. The atom or group of atoms replacing the hydrogen atom is a “substituent.” It should be understood that the terms “substituent”, “substituents”, “moiety”, “moieties”, “group”, or “groups” refer to substituent(s).

The various hydrocarbon-containing moieties provided herein may be described using a prefix designating the minimum and maximum number of carbon atoms in the moiety, namely “Cₐ-C_{b}”. For example, Cₐ-C_{b} alkyl indicates an alkyl moiety having the integer “a” to the integer “b” number of carbon atoms, inclusive.

The term “alkylene” refers to a branched or unbranched (e.g. linear) saturated hydrocarbon chain, unless specified otherwise. An alkylene group is typically unsubstituted, unless indicated otherwise. An alkylene group typically has 1 to 12 carbon atoms, such as 2 to 10 carbon atoms or 4 to 8 carbon atoms. Representative examples include, but are not limited to, methylene, ethylene (e.g. —CH(CH₃)— and —CH₂CH₂—), propylene (e.g. —CH(CH₃)CH₂— and —CH₂CH₂CH₂—) etc.

As used herein, the term “alkyl”, whether used alone or in conjunction with another term, refers to a branched or unbranched saturated hydrocarbon chain, unless specified otherwise. An alkyl group is typically unsubstituted, unless indicated otherwise. An alkyl group may have 1 to 32 carbon atoms, such as 5 to 20 carbon atoms, particularly 1 to 10 carbon atoms. Representative examples include, but are not limited to, methyl, ethyl, n-propyl, i-propyl, n-butyl, i-butyl, s-butyl, t-butyl, n-pentyl, n-hexyl, n-heptyl, n-octyl, n-nonyl, n-decyl, isopropyl, tert-butyl, isobutyl, etc.

The expression “mono-alcohol” as used herein refers to an alcohol having only one alcohol group i.e. having only one hydroxyl (—OH) functional group. In general, the alcohol group is directly bonded to a carbon atom in an aliphatic group. The aliphatic group may include heteroatoms such as oxygen and/or nitrogen, preferably oxygen.

As used herein, the term “glycol” refers to a compound containing two or more hydroxyl (—OH) groups. The term “diol” may also be used to refer to a glycol that contains two hydroxyl groups.

The expression “direct bond” or “directly attached” as used herein means that a group, moiety or substituent is adjacent (i.e. immediately adjacent) and covalently bonded to an atom of another group, moiety or substituent. Put another way, there is no intervening atom, group, moiety or substituent.

The term “average” as used herein in the context of the number-average molecular weight (Mₙ) and the weight-average molecular weight (M_{w}) is the mean. The number-average molecular weight (Mₙ) and the weight-average molecular weight (M_{w}) can be determined by conventional methods, such as gel permeation chromatography (GPC) using, for example, polystyrene as a standard.

Amounts of a material, such as a compound, composition or an ingredient, are typically defined in terms of the wt % of the composition.

The term “about” when used herein in conjunction with a measurable value, such as pH value or temperature, encompasses reasonable variations of the value, for instance, to allow for experimental error in the measurement of said value.

For the avoidance of doubt, the expression “consists essentially of” as used herein limits the scope of a feature to include the specified materials, and any other materials or steps that do not materially affect the basic and novel characteristics of that feature, such as, for example, minor impurities. The expression “consists essentially of’ embraces the expression “consisting of”.

### Citric Acid

Citric acid is used in the preparation of the polyester disclosed herein. Citric acid is a common material of natural origin and has the following structure:

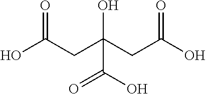 [SMILES: O=C(O)CC(O)(CC(=O)O)C(=O)O]

Citric acid is commercially available, or may be prepared using known synthetic methods, for example through fermentation using a culture of Aspergillus niger that is fed on a sucrose or glucose-containing medium. In making the polyester disclosed herein, citric acid is reacted with one or more mono-alcohols and one or more glycols.

### Mono-Alcohol

The polyester disclosed herein is obtained or obtainable by the reaction of a mono-alcohol with citric acid and a glycol as defined herein. A mono-alcohol or a mixture of mono-alcohols may be used in the reaction. The mono-alcohol may be linear or branched. Preferably, the mono-alcohol is linear. The mono-alcohol may be unsubstituted or substituted. Preferably, the mono-alcohol is unsubstituted. Preferred mono-alcohols include fatty alcohols, which are monohydric aliphatic alcohols and may also be known as “higher” alcohols due to the number of carbon atoms that they contain.

The mono-alcohol is a C₇-C₃₂ mono-alcohol, i.e. the mono-alcohol includes between 7 and 32 carbon atoms. Preferably, the mono-alcohol is a C₁₀ to C₂₅ mono-alcohol, for example a C₁₅ to C₂₀ mono-alcohol.

The mono-alcohol may be a linear fatty alcohol according to the following structure HO—(CH₂)_{b}—CH₃ wherein b is an integer ranging from 6 to 31, for example from 9 to 24, for example from 14 to 19.

Examples of suitable mono-alcohols include enanthic alcohol (C₇), 2-octanol or capryl alcohol (C₈), capric alcohol or decyl alcohol (C₁₀), undecyl alcohol (C₁₁), lauryl alcohol (C₁₂), tridecyl alcohol (C₁₃), myristyl alcohol (C₁₄), pentadecyl alcohol (C₁₅), cetyl alcohol or palmityl alcohol (C₁₆), palmitoleyl alcohol (C₁₆), heptadecyl alcohol (C₁₇), stearyl alcohol (C₁₈), oleyl alcohol (C₁₈), nonadecyl alcohol (C₁₉), octyldodecanol or arachidyl alcohol (C₂₀), heneicosyl alcohol (C₂₁), behenyl alcohol (C₂₂), erucyl alcohol (C₂₂), lignoceryl alcohol (C₂₄), cetryl alcohol (C₂₆), montanyl alcohol (C₂₈), myricyl alcohol (C₃₀), lacceryl alcohol (C₃₂), or mixtures thereof. Preferred alcohols may include lauryl alcohol, stearyl alcohol, and oleyl alcohol. Preferably, the mono-alcohol is lauryl alcohol.

Advantageously, the mono-alcohol may be selected from one or more of those listed below. MW refers to the molecular weight of the mono-alcohols.

| Type | Common name | Carbon atom | MW |
|---|---|---|---|
| Mono | Enanthic alcohol | 7 | 116 |
| alcohol | Capryl alcohol | 8 | 130 |
| | Capric alcohol | 10 | 158 |
| | Lauryl alcohol | 12 | 186 |
| | Myristyl alcohol | 14 | 214 |
| | Pentadecyl alcohol | 15 | 229 |
| | Cetyl alcohol | 16 | 243 |
| | Stearyl alcohol | 18 | 269 |
| | Arachidyl alcohol | 20 | 297 |
| | Cetryl alcohol | 26 | 381 |
| | 2-octanol | 8 | 130 |
| | Octyldodecanol | 20 | 299 |

The mono-alcohols may be naturally available, for example the mono-alcohols may be derived from natural fats and oils. Alternatively, the mono-alcohols used herein may be obtained commercially, or may be prepared using known synthetic methods, for example via transesterification of triglycerides to provide methyl esters, which may then be hydrogenated to produce fatty alcohols.

### Glycol

The polyester disclosed herein is obtained or obtainable by the reaction of a dipropylene glycol or polypropylene glycol, or mixtures thereof, with citric acid and a mono-alcohol as defined herein. The dipropylene glycol or polypropylene glycol as disclosed herein is a diol, in view of the presence of two alcohol groups. In addition, a feature of the dipropylene glycol or polypropylene glycol as used herein is that it contains at least one ether group, for example one ether group (in the case of dipropylene glycol) or from 2 to 34 ether groups in the case of polypropylene glycol as defined by

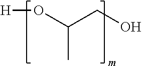

wherein m is from 3 to 35. The presence of an ether group differentiates dipropylene glycol or polypropylene glycol from 1,3-propanediol, which is a diol that does not include an ether group. For simplicity, the dipropylene glycol or polypropylene glycol used to make the polyester may be referred to in some instances as “glycol”.

Preferably, the glycol is dipropylene glycol. Dipropylene glycol has the chemical formula C₆H₁₄O₃ and may be defined by the following structure:

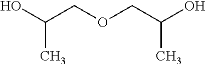

Where dipropylene glycol is defined by

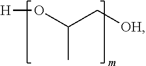

m is 2. Dipropylene glycol may be obtained commercially, or may be prepared using known synthetic methods. Dipropylene glycol may comprise a mixture of three structurally isomeric chemical compounds, namely 4-oxa-2,6-heptandiol, 2-(2-hydroxy-propoxy)-propan-1-ol, and 2-(2-hydroxy-1-methyl-ethoxy)-propan-1-ol.

Generally, polypropylene glycol has the following structure:

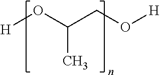

where n is from 3 to 35. Preferably, n is from 5 to 30, more preferably from 10 to 25, for example from 15 to 20. A preferred polypropylene glycol is tripropylene glycol.

The polypropylene glycol may additionally be characterised by its weight average molecular weight. For example, the polypropylene glycol may have a weight average molecular weight of from between about 190 to about 2000, preferably from about 400 to about 1500, for example from about 700 to about 1000. Examples of suitable polypropylene glycols include PPG 400, PPG 700, PPG 1000, and PPG 2000.

The polypropylene glycol may be a polydisperse polymer, for example may have a polydispersity of from about 1 to about 2.5, preferably from about 1.2 to about 2, more preferably from about 1.5 to 1.8. As used herein, the term “polydispersity” indicates the degree of the molecular weight distribution of the polymer sample. Specifically, the polydispersity is a ratio, greater than 1, that is equal to the weight average molecular weight divided by the number average molecular weight. Further information regarding polydispersity can be found in “Principles of Polymerization”, pp. 20-24, G. Odian (John Wiley & Sons, Inc., 3^{rd} ed, 1991) Alternatively the polypropylene glycol may be a monodisperse polymer.

Examples of glycols useful in accordance with the present invention are listed below. 1,3-propanediol is included for comparison purposes only. MW refers to the molecular weight of the glycol, and PO refers to the (average) number of propylene oxide units within the glycol.

| Type | Common name | PO | MW |
|---|---|---|---|
| Diol | 1,3-propanediol | — | 76 |
| | Dipropylene glycol | 2 | 134 |
| | Tripropylene glycol | 3 | 192 |
| | PPG400 | 6.6 | 400 |
| | PPG700 | 11.8 | 700 |
| | PPG1000 | 16.9 | 1000 |
| | PPG2000 | 34.2 | 2000 |

### Polyester

According to an aspect of the invention, there is provided a sunscreen composition comprising a polyester as disclosed herein. The polyester may have a structure as defined in the following section. Additionally or alternatively, the sunscreen composition may comprise a polyester obtained or obtainable by the reaction of a linear or branched C₇-C₃₂ mono-alcohol, or mixtures thereof, citric acid, and a dipropylene glycol or a polypropylene glycol as defined by

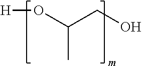

wherein m is from 3 to 35, or a mixture thereof.

According to an aspect of the invention, there is provided a polyester having the following formula:

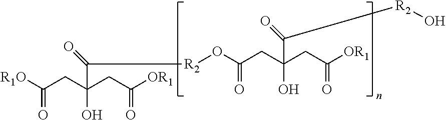

wherein R₁ is a C₇ to C₃₂ linear or branched alkyl group;
R₂ is

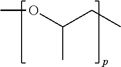

wherein p is from 2 to 35;
n is from 3 to 105.
   R₁ may be a C₁₀ to C₂₅ linear or branched alkyl group, for example a C₁₅ to C₂₀ linear or branched alkyl group. Preferably, R₁ is a linear alkyl group.
   p may be from 4 to 29, more preferably from 9 to 24, for example from 8 to 19.
   n may be from 10 to 100, preferably from 20 to 80, more preferably from 30 to 70, for example from 40 to 60.

The weight average molecular weight of the polyester may range from about 1000 to about 50000, preferably from about 2000 to about 10000, more preferably from about 400 to 5000, for example from about 2500 to about 3500.

### Method

Also disclosed herein is a method of making a polyester. The polyester may be obtained or obtainable by an esterification reaction involving citric acid, a linear or branched C₇-C₃₂ mono-alcohol, or a mixture of said mono-alcohols, and dipropylene glycol or a polypropylene glycol as defined by

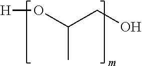

wherein m is from 3 to 35, or a mixture of said dipropylene glycol or polypropylene glycols as defined herein. The method may also be used to form the polyester defined in the previous section. Generally, the method of making the polyester comprises (1) reacting citric acid with one or more linear or branched C₇-C₃₂ mono-alcohols to form an intermediate structure or structures; and (2) reacting the intermediate structure with at least one of a dipropylene glycol or a polypropylene glycol

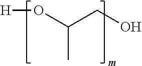

wherein m is from 3 to 35 to produce a polyester. For example, the method may comprise reacting a linear mono-alcohol with citric acid; followed by the addition of dipropylene glycol to form a polyester. An exemplary reaction scheme is provided below:

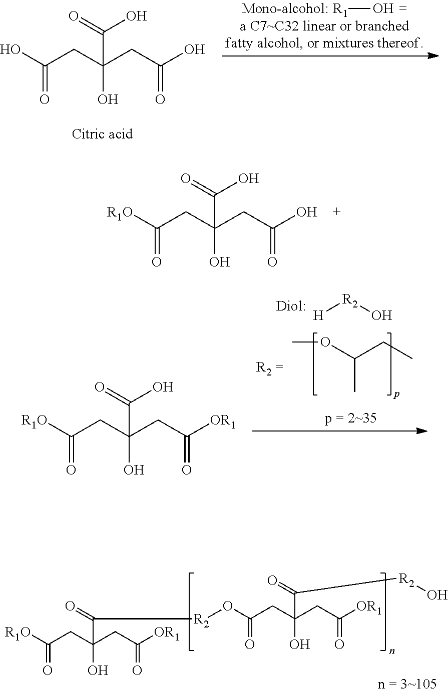

As shown in the exemplary reaction scheme provided above, a mixture of intermediate polyester structures may be formed during the reaction.

In the method according to the invention, advantageously, the citric acid and mono-alcohol are combined first, followed by addition of the glycol. Alternatively, the citric acid, mono-alcohol and glycol are combined simultaneously.

The reaction of citric acid and mono-alcohol may be performed at an elevated temperature, such as from about 120° C. to about 200° C., for example from about 140° C. to about 170° C. The reaction of citric acid and mono-alcohol may be performed at any suitable pressure, for example atmospheric pressure. The molar stoichiometric ratio of citric acid and mono-alcohol is preferably 1:0.9 to 1:2, preferably 1:1 to 1:1.5, for example 1:1. A catalyst such as phosphoric acid may be used. The reaction may be performed under a nitrogen environment. The reaction of citric acid and mono-alcohol may be monitored for a certain period of time, for example by monitoring the acid value, which decreases during the course of the reaction due to citric acid being depleted. During this period, the temperature may be maintained at an elevated temperature. Monitoring of the acid value may be performed by any suitable method, for example, through use of an appropriate indicator.

Once the acid value has stopped decreasing, the glycol may be added. The molar stoichiometric ratio of glycol to the intermediate structure or structures may be 1.1:1 to 0.5:1 for example 1:1. At this point, the temperature of the reaction mixture may be increased further, for example from about 140° C. to about 230° C., such as from about 150° C. to about 200° C. This temperature may be maintained whilst monitoring the acid value, until the acid value fails to decrease.

To provide the polyester, water may be distilled off under vacuum conditions. Optionally, purification of the crude product may be performed. Alternatively, the polyester product may be used without purification.

### Compositions

Also disclosed herein is the use of a polyester in accordance with the invention in a cosmetic composition or a personal care composition. A cosmetic composition may be understood to refer to a composition to enhance and/or beautify the appearance of a person. A personal care composition may be understood to encompass grooming and personal hygiene products. The terms “cosmetic composition” and “personal care composition” may also be used interchangeably. A cosmetic composition or personal care composition as defined herein may be applied to the exterior of the human body, for example to the skin or hair. The composition may be a leave-on composition. A “leave-on” composition refers to a composition that is applied to the exterior of the human body, and remains in situ for an amount of time without being washed off by the user, for example a sunscreen composition. Alternatively, the composition may be a rinse-off composition. A “rinse-off” composition refers to a composition that is applied to the exterior of the human body, and is washed off after a certain period of time.

Non-limiting examples of cosmetic compositions and personal care compositions may include fragrance, make-up cosmetics, skin-care cosmetics, hair-care products and special-purpose cosmetics. Examples of fragrance include perfume or eau de cologne. Examples of make-up cosmetics include foundation e.g. foundation creams, lipsticks, and eye make-up. Examples of skin-care cosmetics include facial cream, skin lotion, skin milk and cleansing cream. Examples of hair care products include hair dye, shampoo, and hair treatments. Examples of special purpose cosmetics include sunscreen.

The polyester may be used in a film-forming composition. Preferably, the polyester may be included in a sunscreen composition.

The sunscreen composition typically comprises an amount of about 0.1 to about 5.0 wt % of the polyester, preferably an amount of about 0.5 to about 4.0 wt %, more preferably an amount of about 1.0 wt % to about 3.0 wt %, for example an amount of about 1.5 wt % to 2.0 wt % of the sunscreen composition. In general, the sunscreen composition comprises an amount of polyester that can provide effective film-forming properties.

Preferably, the sunscreen composition may comprise at least one UV filter. The UV filter may be a physical filter, such as a mineral filter, which acts as a physical barrier that prevents some or all UV radiation from reaching the skin. The UV filter may be a chemical filter, which can absorb some or all of the UV radiation arriving at the skin. Examples of suitable UV filters include titanium dioxide, zinc oxide, octocrylene, homosalate, 4-methylbenzylidene camphor, benzophenone-3, benzophenone-4, avobenzone, bis-ethylhexyloxyphenol methoxyphenyl triazine, butyl methoxydibenzoylmethane, diethylamino hydroxybenzoyl hexyl benzoate, caprylyloxyphenylamino dimethyltetrahydro benzothiazine carboxylic acid, diethylhexyl butamido triazone, drometrizole trisiloxane, ethylhexyl methoxycinnamate, ethylhexyl salicylate, phenylbenzimidazole sulfonic acid, terephthalylidene dicamphor sulfonic acid, methylene bis-benzotriazolyl tetramethylbutylphenol, ethylhexyl triazone, sodium phenylbenzimidazole sulfonate, isoamyl p-methoxycinnamate, disodium phenyl dibenzimidazole tetrasulfonate and mixtures thereof. Preferred UV filters may include octocrylene, ethylhexyl methoxycinnamate, ethylhexyl salicylate, homosalate, avobenzone, and mixtures thereof. Preferably, the composition comprises both physical and chemical filters. The UV filter or filters may be present in the sunscreen composition in a total amount of from about 2 wt % to about 35 wt %, preferably from about 5 wt % to 30 wt %, more preferably from about 10 wt % to about 25 wt %, for example from about 15 wt % to about 20 wt %.

Typically, the sunscreen composition may contain further additives. Examples of additives include emollients, humectants, skin conditioners, vitamins, moisturizers, pigments, viscosity controlling agents, biological actives, extracts, preservatives, emulsifiers, antioxidants, light stabilizers, film formers, opacifiers, vitamins, pigments and skin protectants. One or more of these additives may be included in the sunscreen composition. Each of said further additives may individually be present in the sunscreen composition in an amount of from about 0.5 wt % to about 10 wt %, preferably from about 1 wt % to about 5 wt %, for example from about 2 wt % to about 3 wt %.

Preferably, the sunscreen composition does not comprise microplastic particles, i.e. is essentially free of microplastic particles. For example, the sunscreen composition preferably does not comprise microbeads which are solid plastic particles that may have an mean average size (longest dimension of the microbead) of less than 1 mm.

The format of the sunscreen composition is not particularly limited. The polyester disclosed herein may be used in a wide range of sunscreen compositions, such as water/oil (W/O) emulsions, oil/water (O/W) emulsions, silicone/water (S/W) emulsions, alcohol-based systems and anhydrous formulations. Preferably, the sunscreen composition is an oil/water emulsion or an alcohol-based system such as an ethanol-based system.

The sunscreen compositions may be applied via any suitable format. The sunscreen composition may be applied by means of a spray. Alternatively, the sunscreen composition may be applied directly to the skin by hand, particularly when the sunscreen is in the form of a lotion or cream. Alternatively, the sunscreen composition may be in stick format, for example, wherein the composition is in anhydrous form.

Generally, the sunscreen composition comprises a solvent phase. The solvent phase may include water, ethanol, a hydrophobic carrier oil, or a mixture thereof. Exemplary solvents include caprylic/capric triglyceride, octyldodecanol, C12-15 alkyl benzoate, dimethicone, phenyl trimethicone, cyclopentasiloxane, isododecane, dibutyl adipate, olea Europaea fruit oil, butyloctyl salicylate, isohexadecane, isopropyl palmitate, isopropyl myristate, glycerin, butylene glycol, propanediol, and mixtures thereof. The solvent may be present in the sunscreen composition in an amount of from about 15 wt % to about 60 wt %, preferably from about 10 wt % to about 50 wt %, more preferably from about 15 wt % to about 40 wt %, for example from about 20 wt % to about 30 wt %.

Advantageously, the sunscreen composition may be an emulsion, for example a water/oil (W/O) emulsion, an oil/water (O/W) emulsion, or a silicone/water (S/W) emulsion. In an emulsion in accordance with the invention the aqueous phase may include (in addition to water) humectants, UV filters, skin conditioners, vitamins, moisturizers, pigments, viscosity controlling agents, pigments, biological actives, extracts, water soluble colorants, preservatives or mixtures thereof.

Where the emulsion includes an oil phase, the oil phase may include solvents, emulsifiers, skin conditioners, antioxidants, light stabilizers, viscosity controlling agents, film formers, opacifiers, UV filters, vitamins, pigments, skin protectants, or mixtures thereof.

### EXAMPLES

The invention is illustrated by the following non-limiting examples.

Examples 1 to 7 detail polyester synthesis using the raw materials and ratios listed in Table 1 below. Table 1 also details the raw materials and ratios used in Comparative Examples 1 and 2.

### Example 1—Polyester Synthesis

A glass reactor equipped with an overhead stirrer, a thermocouple, a dean-stark trap, and a condenser was prepared. The reactor was charged with 0.5 mole of citric acid, 0.5 mole of lauryl alcohol, and phosphoric acid under agitation. The reaction mixture was heated to 140˜160° C. in a nitrogen environment Water was distilled from the reaction mixture. The reaction was monitored by determination of the acid value of the reaction mixture, and the reaction temperature was kept at a constant temperature until the acid value failed to decrease. 0.6 mole of dipropylene glycol was then added to the reaction mixture, and the reaction mixture was heated to 170˜230° C. Water was distilled off under vacuum conditions. The reaction mixture was kept at constant temperature until the acid value failed to decrease. Then reaction mixture was then cooled. The crude product was then used without purification.

### Examples 2 to 7—Polyester Synthesis

Further polyesters were obtained using the synthesis process described in Example 1. Table 1 lists the materials and ratios used in Examples 2 to 7.

### Comparative Examples 1 and 2

Comparative Examples 1 and 2 relate to forming polyesters that are not in accordance with the present invention. The polyesters of Comparative Examples 1 and 2 were obtained using the synthesis process of Example 1. However, instead of dipropylene glycol or a polypropylene glycol (such as tripropylene glycol or PPG 400), 1,3-propanediol was used.

TABLE 1

| | | | | | | | | Polyester | |
|---|---|---|---|---|---|---|---|---|---|
| | | | | | | | | synthesis | |
| Raw | Polyester synthesis | | | | | | | Comparative | |
| materials | Examples | | | | | | | Examples | |
| (mole) | 1 | 2 | 3 | 4 | 5 | 6 | 7 | 1 | 2 |
| Lauryl alcohol | 0.5 | 1 | 0.8 | 0.4 | 0.4 | 0.6 | 0.6 | 0.8 | 0.8 |
| 2-octanol | | | | 0.4 | 0.4 | | | | |
| Octyldodecanol | | | | 0.03 | | | | | 0.05 |
| 1,3-propanediol | | | | | | | | 0.4 | 0.4 |
| Dipropylene glycol | 0.6 | 0.3 | 0.4 | 0.4 | 0.4 | | | | |
| Tripropylene glycol | | | | | | 0.3 | | | |
| PPG400 | | | | | | | 0.3 | | |
| citric acid | 0.5 | 0.5 | 0.5 | 0.5 | 0.5 | 0.4 | 0.4 | 0.5 | 0.5 |

### Formulation of Sunscreen Compositions

Oil/water formulations were prepared based on the polyesters of Examples 1 to 7 and Comparative Examples 1 and 2. Details of the formulation is provided in Table 2 below, where the film former of Phase B is the polyester of Examples 1 to 7 and Comparative Examples 1 and 2. The calculated SPF of the oil/water formulations was 30. The pH of the formulations was adjusted to 6.2 to 6.5.

TABLE 2

| | | | | 2% |
|---|---|---|---|---|
| | | | Control | sample |
| | INCI | Trade Name | w/w % | w/w % |
| Phase | Octocrylene | Eusolex OCR | 8 | 8 |
| A | Ethylhexyl | Eusolex 2292 | 5 | 5 |
| | Methoxycinnamate | | | |
| | Homosalate | Eusolex HMS | 5 | 5 |
| | Ethylhexyl Salicylate | Eusolex OS | 5 | 5 |
| | BMDBM (Avobenzone) | Eusolex 9020 | 2.8 | 2.8 |
| | Dicaprylyl Carbonate | Cetiol CC | 8.4 | 6.4 |
| Phase | Film Former | | 0 | 2 |
| B | | | | |
| Phase | Polyglyceryl-6 Stearate | Tego Care PBS 6 | 3 | 3 |
| C | (and) Polyglyceryl-6 | | | |
| | Behenate | | | |
| | Cetearyl Alcohol | Tego Alkanol | 2 | 2 |
| | | 1618 | | |
| Phase | Water | | 57.8 | 57.8 |
| D | Propandiol | | 2 | 2 |
| | Phenoxyethanol (and) | Euxyl PE 9010 | 1 | 1 |
| | Ethylhexylglycerin | | | |
| Phase | Citric Acid 25% | | q.s. | q.s. |
| E | | | | |
| Total | | | 100 | 100 |

SPF performance tests were performed on formulations containing the polyester of Examples 1 to 7 and Comparative Examples 1 and 2.

### SPF Performance Testing Procedure

Application of product: 32.5 mg of product, which equates to 2 mg/cm² of product, was evenly applied to a PMMA plate (HelioScreen PMMA HD6 (5×5 cm)) using a standardised technique. The product was then allowed to dry for 20 min in the dark. This was needed to avoid extraneous exposure to artificial or natural UV light.

Initial SPF: the initial SPF before washing was determined using the Labsphere UV-2000 device, software version 1.4.6.0, according to ISO 24443-2012.

Irradiation: after determination of the initial SPF, the PMMA plates were exposed to UV light at 313 nm and 1.65 W/m²*nm for 45 seconds with an Atlas UV Test instrument to mimic a solar light simulator. The radiation dosage had an irradiation intensity equivalent to 2.5 MED.

To account for a lack of photostability and to achieve more relevant results, a UV pre-irradiation step at a fixed dose is commonly used in SPF-testing laboratories and in line with literature (S. Miksa et.al. New approach for a reliable in vitro sun protection factor method Part I: Principle and mathematical aspects, International Journal of Cosmetic Science, 2015, 37, 555-566). In irradiation testing, a solar light simulator providing irradiation in the range of 290-800 nm may be used for the pre-irradiation step. The irradiation testing as performed herein was modified by using the Atlas UV Test instrument providing UVB light at 313 nm only.

Water immersion procedure: a glass beaker filled with 750 ml tap water at room temperature (21° C.-23° C.) was placed on a magnetic stirrer. The PMMA plates were fully submersed with water and stirred at 250 rpm for 20 min to guarantee continuous water circulation to simulate moderate activity. After washing, the PMMA plates were removed from the water bath and allowed to air dry for 20 min at room temperature (21° C.-23° C.) without wiping. A second water immersion step was subsequently performed to demonstrate water resistance.

After the last drying step, no water drops should be visible. The final SPF of the product was determined with the Labsphere UV-2000 device (spectrum from 290 nm to 450 nm), software version 1.4.6.0., method: ISO 24443-2012.

The initial SPF, remaining SPF and SPF retention percentages for sunscreen compositions comprising the polyesters of Examples 1 to 7 and Comparative Examples 1 and 2 are presented in Table 3 below. Higher levels of SPF retention were observed for the compositions comprising polyesters of Examples 1 to 7 according to the invention, as compared to Comparative Examples 1 and 2.

TABLE 3

| | | | | | | | | Comparative | |
|---|---|---|---|---|---|---|---|---|---|
| | Polyester synthesis examples | | | | | | | examples | |
| | 1 | 2 | 3 | 4 | 5 | 6 | 7 | 1 | 2 |
| Initial | 14 | 27 | 28 | 52 | 30 | 31 | 18 | 66 | 70 |
| SPF | | | | | | | | | |
| Remaining | 14 | 15 | 27 | 45 | 30 | 25 | 19 | 41 | 51 |
| SPF | | | | | | | | | |
| SPF | 99 | 56 | 96 | 88 | 100 | 86 | 109 | 62 | 73 |
| retention | | | | | | | | | |

### Water Retention

The above-described procedure is based on ISO 16217 (Water Resistance in vivo July 2020), incorporating requirements from COLIPA (Cosmetics Europe), FDA Monograph 2011 and Australia (AS/NZS 2604:2012).

The water immersion procedure may be repeated to measure a water resistant claim (i.e. two water immersion steps) before measurement of the remaining SPF. Four water immersion steps can be carried out to provide a very water resistant claim.

Water resistance retention may be determined as follows:

$$\% Water Resistance Retention ( \% WRR ) = 100 / ( initial SPF ) * ( remaining SPF )$$

initial SPF: measured value after application and 20 min drying step
remaining SPF: measured value after washing procedure (either for water resistant or very water resistant procedure)

FIG. 1 shows the film integrity on PMMA plates after washing. In FIG. 1, RD23620 indicates the polyester according to Example 4 (Table 1) incorporated in the formulation according to Table 2. Improved water resistance was shown for RD23620 after 2×20 minutes washing in comparison to a control sample (formulation that does not comprise a film former according to the invention) and a commercially-available film former (SolAmaze natural). Good water resistance was also observed after 4×20 minutes washing. This demonstrates both a “water resistant claim” and “very water resistant claim” as defined herein.

FIG. 2 shows that RD23620 shows a SPF boost and improved water resistance when compared to both the control and competitive film former sample. On washing for both 2×20 minutes and 4×20 minutes, more than 50% of the initial SPF is retained for RD23620.

FIGS. 3 and 4 provide further illustration of film integrity and SPF performance for formulations in accordance with the invention, namely formulations comprising the film former polyester of Examples 6 and 7 after 2×20 minutes washing.

### Alcohol-Based System

Film former RD23620 was also tested as to compatibility in an alcohol-based composition. Details of the composition are as follows:

| INCI | Trade Name | Wt % |
|---|---|---|
| Alcohol | SD Alcohol 40B | 80 |
| | 190 | |
| Octisalate | Eusolex OS | 5 |
| Octocrylene | Eusolex OCR | 7 |
| Oxybenzone | Eusolex 4360 | 3 |
| Film Former | RD 23620 | 5 |

FIG. 5 shows that RD23260 shows good compatibility with an alcohol system. As shown, RD23260 (ii) shows the same clear appearance as the control sample (i) (no film former). This can be compared with a composition containing a polyester not in accordance with the invention (iv) (TegoFilm Star One MB), which shows increased instability with precipitation, and a citric acid-based polyester with 1,3 propanediol (iii), where turbidity is seen.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

The entire disclosure of each document cited herein is hereby incorporated herein by reference. All references cited in this disclosure are incorporated by reference to the same extent as if each reference had been incorporated by reference in its entirety individually.

The citation and incorporation of patent documents herein is done for convenience only and does not reflect any view of the validity, patentability, and/or enforceability of such patent documents.

A number of embodiments of the disclosure have been described. Nevertheless, it will be understood that various modifications may be made without departing from the spirit and scope of the present disclosure. Accordingly, other embodiments are within the scope of the following claims.

All headings and sub-headings are used herein for convenience only and should not be construed as limiting the invention in any way.

The use of any and all examples, or exemplary language (e.g. “such as”) provided herein, is intended merely to better illuminate the invention and does not pose a limitation on the scope of the invention unless otherwise paragraphed. No language in the specification should be construed as indicating any non-paragraphed element as essential to the practice of the invention.

1. A polyester obtained or obtainable by the reaction of:
a linear or branched C₇-C₃₂ mono-alcohol, or mixtures thereof;
citric acid; and
dipropylene glycol or a polypropylene as defined by 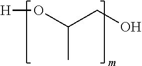
wherein m is from 3 to 35; or mixtures thereof.

2. The polyester of claim 1, wherein the mono-alcohol is a linear fatty alcohol according to the following structure
HO—(CH₂)_{b}—CH₃
wherein b is an integer ranging from 6 to 31.

3. The polyester of claim 2 wherein the alcohol is lauryl alcohol, stearyl alcohol, oleyl alcohol, and mixtures thereof.

4. The polyester of claim 1, wherein the dipropylene glycol or polypropylene glycol is dipropylene glycol.

5. The polyester of claim 1, wherein the polypropylene glycol is selected from tripropylene glycol, PPG 400, PPG 700, PPG 1000, PPG 2000, and mixtures thereof.

6. A polyester having the following formula: 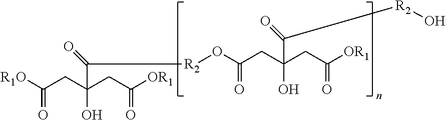
wherein R₁ is a C₇ to C₃₂ linear or branched alkyl group;
R₂ is 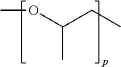
p is from 2 to 35; and
n is from 3 to 105.

7. (canceled)

8. A sunscreen composition comprising the polyester of claim 1.

9. The sunscreen composition of claim 8, wherein the composition further comprises at least one UV filter, wherein the UV filter is optionally selected from titanium dioxide, zinc oxide, octocrylene, homosalate, 4-methylbenzylidene camphor, benzophenone-3, benzophenone-4, bis-ethylhexyloxyphenol methoxyphenyl triazine, butyl methoxydibenzoylmethane, diethylamino hydroxybenzoyl hexyl benzoate, caprylyloxyphenylamino dimethyltetrahydro benzothiazine carboxylic acid, diethylhexyl butamido triazone, drometrizole trisiloxane, ethylhexyl methoxycinnamate, ethylhexyl salicylate, phenylbenzimidazole sulfonic acid, terephthalylidene dicamphor sulfonic acid, methylene bis-benzotriazolyl tetramethylbutylphenol, ethylhexyl triazone, sodium phenylbenzimidazole sulfonate, isoamyl p-methoxycinnamate, disodium phenyl dibenzimidazole tetrasulfonate and mixtures thereof.

10. The sunscreen composition of claim 8, wherein the composition further comprises at least one selected from humectants, skin conditioners, vitamins, moisturizers, pigments, viscosity controlling agents, biological actives, extracts, preservatives, emulsifiers, antioxidants, light stabilizers, film formers, opacifiers, vitamins, pigments and skin protectants.

11. The sunscreen composition of claim 8, wherein the composition further comprises a solvent, wherein the solvent is optionally selected from water or at least one selected from caprylic/capric triglyceride, octyldodecanol, C₁₂₋₁₅ alkyl benzoate, dimethicone, phenyl trimethicone, cyclopentasiloxane, isododecane, dibutyl adipate, olea Europaea fruit oil, butyloctyl salicylate, isohexadecane, isopropyl palmitate, isopropyl myristate, glycerin, butylene glycol, propanediol, and mixtures thereof.

12. The sunscreen composition of claim 8, wherein the sunscreen composition is in the form of a spray, lotion, stick or cream.

13. The sunscreen composition of claim 8, wherein the polyester is present in the sunscreen composition in an amount of from 0.1 to about 5.0 wt %.

14. (canceled)
